(19) United States
(12) Patent Application Publication
(10) Pub. No.: US 2014/0294854 A1
(43) Pub. Date: Oct. 2, 2014

(54) TARGET DIRECTED TO ADIPOCYTES, METHODS AND ASSAYS FOR TREATMENT OF OBESITY

(71) Applicants: Streamson Coo Chua, Dobbs Ferry, NY (US); Shun-Mei Liu, Bronx, NY (US); Genevieve Marcelin, New York, NY (US)

(72) Inventors: Streamson Coo Chua, Dobbs Ferry, NY (US); Shun-Mei Liu, Bronx, NY (US); Genevieve Marcelin, New York, NY (US)

(73) Assignee: Albert Einstein College of Medicine of Yeshiva University, Bronx, NJ (US)

(21) Appl. No.: 14/001,685

(22) PCT Filed: Mar. 8, 2012
(86) PCT No.: PCT/US12/28242

§ 371 Date: Sep. 11, 2013

Related U.S. Application Data
(60) Provisional application No. 61/451,361, filed on Mar. 10, 2011.

(51) Int. Cl. C07K 16/40; G01N 33/573; C12N 15/113; C12Q 1/68

(52) U.S. Cl. CPC C07K 16/40; C12Q 1/686; G01N 33/573; C12N 15/1137; A61K 2039/505
USPC 424/146.1; 424/158.1; 435/6.12; 435/7.4; 514/44 A

(57) ABSTRACT

The present invention provides compositions and methods for treating obesity in a subject by administering an inhibitor of UBE2L6 or an activator of adipocyte triglyceride lipase to the subject.

### CROSS-REFERENCE TO RELATED APPLICATIONS

This application claims benefit of U.S. Provisional Application No. 61/451,361, filed Mar. 10, 2011, the contents of which are hereby incorporated by reference.

### STATEMENT OF GOVERNMENT SUPPORT

This invention was made with government support under grant number DK057621 awarded by the National Institute on Aging, National Institutes of Health, U.S. Department of Health and Human Services. The government has certain rights in the invention.

### FIELD OF THE INVENTION

The present invention relates generally to treating obesity through manipulating triglyceride release from fat cells.

### BACKGROUND OF THE INVENTION

Throughout this application various publications are referred to by number in parenthesis. Full citations for these references may be found at the end of the specification. The disclosures of these publications, all books and all patents and patent application publications referred to herein are hereby incorporated by reference in their entirety into the subject application to more fully describe the art to which the subject invention pertains.

Obesity is the accumulation of excess triglycerides within adipocytes, increasing body fat content. Leptin is the major hormonal regulator of body fat and defective leptin signaling within the hypothalamus is a major factor in the development of diet induced obesity (1). Due to the difficulty of accessing and manipulating hypothalamic neurons, alternative means of circumventing the leptin signaling defect would be a significant aid in identifying effective treatments for obesity.

The obesity epidemic has reached global proportions and there are few effective therapeutic approaches (9). An alarming trend has been observed in pediatric populations with increasing rates of overweight and obese children over the past two to three decades (10). As obesity is a chronic disease with increasing risks associated with chronicity, it is likely that the obese pediatric population will age into an obese adult population with more severe complications. While acknowledging that many of the causes of obesity are related to societal changes, it remains an uncomfortable fact that obese individuals will require treatment to minimize the impact of obesity and its associated co-morbidities of type 2 diabetes mellitus and cardiomegaly/cardiomyopathy (11).

Current treatment modalities include behavioral modification, drug therapy and various forms of gastric bypass. All of these treatments are designed to produce weight loss in both fat mass and fat-free mass—there is an associated loss of skeletal muscle mass which should be deemed undesirable. A targeted treatment at decreasing fat mass without losing lean mass remains elusive. The present application addresses this need.

### SUMMARY OF THE INVENTION

A method for treating obesity, treating an obesity comorbidity, or treating a dyslipidemia in a subject comprising administering to the subject an amount of an inhibitor of an E2 ubiquitin ligase activity effective to treat obesity, obesity comorbidity, or dyslipidemia.

A method for treating obesity or a dyslipidemia in a subject comprising administering to the subject an amount of an activator of adipocyte triglyceride lipase, or an enhancer of adipocyte triglyceride lipase activity, effective to treat obesity or dyslipidemia.

A method for reducing body weight in a subject without decreasing lean muscle mass comprising administering to the subject an amount of an inhibitor of an E2 ubiquitin ligase activity effective to reduce body weight in a subject without decreasing lean muscle mass.

A method for identifying an agent as a treatment for obesity or as a candidate agent for treating obesity comprising:

a) contacting a protein with UBE2L6 under conditions permitting ubiquitination of the protein by the UBE2L6;
b) quantitating the ubiquitination of the protein by the UBE2L6;
c) contacting the UBE2L6 with the agent; and
d) quantitating the ubiquitination of the protein by the UBE2L6 in the presence of the agent,
wherein a decreased ubiquitination of the protein by the UBE2L6 in the presence of the agent as compared to in the absence of the agent indicates that the agent is a treatment for obesity or is a candidate agent for treating obesity and wherein no change in or an increased ubiquitination of the protein by the UBE2L6 in the presence of the agent as compared to in the absence of the agent indicates that the agent is not a treatment for obesity or is not a candidate agent for treating obesity.

A method for identifying an agent as a treatment for obesity or as a candidate agent for treating obesity comprising:

a) quantitating UBE2L6 conjugation with ISG1.5 in a sample;
b) contacting the sample comprising the UBE2L6 with the agent; and
c) quantitating UBE2L6 conjugation with ISG15 in the sample in the presence of the agent,
wherein an increased UBE2L6 conjugation with ISG15 in the presence of the agent as compared to in the absence of the agent indicates that the agent is a treatment for obesity or is a candidate agent for treating obesity and wherein no change in or a decreased UBE2L6 conjugation with ISG15 in the presence of the agent as compared to in the absence of the agent indicates that the agent is not a treatment for obesity or is not a candidate agent for treating obesity.

An agent identified by any of the instant methods.

An inhibitor of E2 ubiquitin ligase activity for treating obesity in a subject.

An activator of adipocyte triglyceride lipase, or an enhancer of adipocyte triglyceride lipase activity, for treating obesity in a subject.

A composition comprising (i) an antibody, or a fragment of an antibody, which antibody or fragment binds to UBE2L6 and inhibits UBE2L6 ubiquitination activity, or (ii) an shRNA or siRNA which inhibits expression of UBE2L6, or (iii) a small molecule inhibitor of E2 ubiquitin ligase.

A pharmaceutical composition comprising (i) an antibody or a fragment of an antibody, which antibody or fragment binds to UBE2L6 and inhibits UBE2L6 ubiquitination activity, or (ii) a shRNA or siRNA which inhibits expression of UBE2L6, or (iii) a small molecule inhibitor of E2 ubiquitin ligase, and a pharmaceutically acceptable carrier.

A method for treating obesity, treating an obesity comorbidity, or treating a dyslipidemia in a subject comprising administering to the subject an amount of an enhancer of UBE2L6 conjugation with ISG15 effective to treat obesity, obesity comorbidity, or dyslipidemia.

### BRIEF DESCRIPTION OF THE DRAWINGS

FIG. 1. The role of ISG15 in reducing ubiquitination by withdrawing UBE2L6 from the ubiquitination cycle. The E1 enzyme catalyzes ubiquitin or ubiquitin like peptide (Ubl) activation, resulting in formation of an E1-Ub(l) intermediate. The activated E1 transfers the Ub/Ubl to the active site cysteine of the E2 enzyme. E3s function as scaffolding to bind targeted proteins and activated E2, orienting them for Ub/Ubl conjugation to the amino group of a lysine in the substrate. Each of the enzymes are generally specific for Ub or Ubl although UBE2L6 can use either ubiquitin or ISG15.

FIGS. 2A-2B. Increased fatty acid oxidation is associated with decreased white adipocyte cell size. (2A) Respiratory exchange ratios (RER) were determined during the light and dark cycles in BALB/c, B6 and F1 ob/ob mice with free access to breeder chow (n=4 mice/group; data averaged over 5 days). (2B) Morphology (H&E) of white adipose tissue of 3-month old BALB/c and B6 ob/ob mice (magnification ×20 for WAT). Data are expressed as average±SEM. Two-way ANOVA (2A) and unpaired t-tests (2B) were performed, *P<0.05 compared to BALB/c.

FIG. 3. Increased adipose lipolysis in BALB/c ob/ob mice. Glycerol release from explants of fresh inguinal WAT of BALB/c and B6 ob/ob mice under basal conditions or stimulated with isoproterenol (n=6). Data are expressed as average ±SEM. Unpaired t-tests were performed, *P<0.05 compared to BALB/c ob/ob in the same condition.

FIGS. 4A-4C. Accumulation of adipose triglyceride lipase (“ATGL”) and CGI-58 in WAT of BALB/c ob/ob mice. Immunoblots of (4A) HSL, (4B) Perilipin A, (4C) ATGL and CGI-58 in adipose tissue of BALB/c, B6 and F1 ob/ob mice. Densitometry data are expressed as average ±SEM (n=6). Unpaired t-tests were performed, *P<0.05 compared to BALB/c ob/ob.

FIGS. 5A-5C. Regulation of ATGL expression through degradation pathways. (5A) ATGL mRNA level in WAT was determined by RT-qPCR (n=6). (5B) WAT fragments of BALB/c and B6 ob/ob mice were incubated for 5 hours with cycloheximide (CHX) and WAT protein lysates were immunoblotted using anti-ATGL to follow degradation rates of ATGL. (5C) B6 ob/ob WAT fragments were treated with MG132 (MG, proteasome inhibitor) and chloroquine (CQ, lysosome inhibitor). Protein extracts were analyzed by Western blot. Densitometry analysis was performed (5B,5C) and data are expressed as average ±SEM (n=6). Unpaired t-tests were performed, *P<0.05.

FIGS. 6A-6B. Markers on Chr 2 are associated with obesity resistance phenotype of BALB/c ob/ob mice. (6A) Fat mass % distribution at 3 month-old of B6, BALB/c, F1 and N2 ob/ob mice. (6B) Diagram of fat mass percentage of N2 mice by their haplotype on chromosome 2 and 3. CC designates the homozygous state of BALB/c alleles and BC is for heterozygous state of BALB/c and B6 alleles. Data are expressed as average ±SEM (n=6). Unpaired t-tests were performed, *P<0.05.

FIGS. 7A-7D. Variation of Ubc216—coding sequences and expression in BALB/c and C57BL/6. (7A) Electrophoregrams of the B6 and BALB/c alleles of Ube216 showing polymorphism that affect codon 28: ASP (D) in B6 and TYR (Y) in BALB/c. (7B) Immunoblot and densitometry with anti-UBE2L6 of whole protein extract from ob/ob BALB/c (7C), B6 (7B) and F1 WAT. Asterisks denote a significant difference (p<0.05) between groups in pairwise analyses. (7C) Immunoblot and densitometry with anti-ISG15 of whole protein extract from BALB/c (7C), B6 (7B) and F1 fat pad. Asterisks denote a significant difference (p<0.05) between groups in pairwise analyses. (7D) Body weight (BW) and fat mass of the recombinant congenic ob/ob BALB/c with chromosome 2 heterozygous for B6 and BALB/c alleles. Data are expressed as average ±SEM (n=6). Unpaired t-tests were performed, *P<0.05.

FIG. 8. A molecular mechanism that describes the strain specific differences in body fat content of BALB/c and C57BL/6 ob/ob mice. Font sizes and arrows are drawn in proportion to the steady state concentrations of the various molecules and processes, respectively. ATGL—adipose triglyceride lipase; E2—E2 ubiquitination enzyme; E2-S-Ub—activated E2 with thioester bond to ubiquitin; E2-ISG15—ISIS conjugated E2; E3—E3 ubiquitination enzyme; Ub-ATGL—ubiquitinated ATGL.

### DETAILED DESCRIPTION OF THE INVENTION

A method is provided for treating obesity, treating an obesity comorbidity, or treating a dyslipidemia in a subject comprising administering to the subject an amount of an inhibitor of an E2 ubiquitin ligase activity effective to treat obesity, obesity comorbidity, or dyslipidemia.

Also provided is a method for treating obesity or a dyslipidemia in a subject comprising administering to the subject an amount of an activator of adipocyte triglyceride lipase, or an enhancer of adipocyte triglyceride lipase activity, effective to treat obesity or dyslipidemia.

Also provided is a method for reducing body weight in a subject without decreasing lean muscle mass comprising administering to the subject an amount of an inhibitor of an E2 ubiquitin ligase activity effective to reduce body weight in a subject without decreasing lean muscle mass.

In an embodiment of the methods, the E2 ubiquitin ligase is UBE2L6. In an embodiment of the methods, the inhibitor of E2 ubiquitin ligase is an antibody. In an embodiment of the methods, the antibody is a monoclonal antibody. In an embodiment of the methods, the inhibitor of E2 ubiquitin ligase is an shRNA or siRNA directed to UBE2L6. In an embodiment of the methods, the enhancer of adipocyte triglyceride lipase activity is a UBE2L6 inhibitor. In an embodiment of the methods, the UBE2L6 inhibitor is an antibody. In an embodiment of the methods, the antibody is a monoclonal antibody. In an embodiment of the methods, the method is for treating obesity.

Also provided is a method for identifying an agent as a treatment for obesity or as a candidate agent for treating obesity comprising:

a) contacting a protein with UBE2L6 under conditions permitting ubiquitination of the protein by the UBE2L6;
b) quantitating the ubiquitination of the protein by the UBE2L6;
c) contacting the UBE2L6 with the agent; and
d) quantitating the ubiquitination of the protein by the UBE2L6 in the presence of the agent,
wherein a decreased ubiquitination of the protein by the UBE2L6 in the presence of the agent as compared to in the absence of the agent indicates that the agent is a treatment for obesity or is a candidate agent for treating obesity and wherein no change in or an increased ubiquitination of the protein by the UBE2L6 in the presence of the agent as compared to in the absence of the agent indicates that the agent is not a treatment for obesity or is not a candidate agent for treating obesity.

Also provided is a method for identifying an agent as a treatment for obesity or as a candidate agent for treating obesity comprising:

a) quantitating UBE2L6 conjugation with ISG15 in a sample;
b) contacting the sample comprising the UBE2L6 with the agent; and
c) quantitating UBE2L6 conjugation with ISG15 in the sample in the presence of the agent,
wherein an increased UBE2L6 conjugation with ISG15 in the presence of the agent as compared to in the absence of the agent indicates that the agent is a treatment for obesity or is a candidate agent for treating obesity and wherein no change in or a decreased UBE2L6 conjugation with ISG15 in the presence of the agent as compared to in the absence of the agent indicates that the agent is not a treatment for obesity or is not a candidate agent for treating obesity.

In an embodiment of the methods, the method is performed in vitro. In an embodiment of the methods, the agent is a small organic molecule of 2,000 daltons or less. In an embodiment of the methods, the agent is a small organic molecule of 800 daltons or less. In embodiments of the methods, the agent is an antibody, an oligonucleotide, an shRNA or an siRNA.

Also provided is an agent identified by any of the instant methods. Also provided is an inhibitor of E2 ubiquitin ligase activity for treating obesity in a subject. Also provided is an activator of adipocyte triglyceride lipase, or an enhancer of adipocyte triglyceride lipase activity, for treating obesity in a subject.

In an embodiment of the methods, agents, or inhibitors, the E2 ubiquitin ligase is UBE2L6. In an embodiment, the inhibitor of E2 ubiquitin ligase is an antibody. In an embodiment, the antibody is a monoclonal antibody. In an embodiment, the inhibitor of E2 ubiquitin ligase is an shRNA or siRNA directed to UBE2L6. In an embodiment, the inhibitor of E2 ubiquitin ligase is a small molecule of 2000 daltons or less. In an embodiment, the small molecule is an organic small molecule.

Also provided is a composition comprising (i) an antibody which binds to UBE2L6 and inhibits UBE2L6 ubiquitination activity, or (ii) an shRNA or siRNA which inhibits expression of UBE2L6 or (iii) a small molecule inhibitor of E2 ubiquitin ligase.

In an embodiment the composition comprises a pharmaceutically acceptable carrier.

Also provided is a pharmaceutical composition comprising (i) an antibody which binds to UBE2L6 and inhibits UBE2L6 ubiquitination activity, or (ii) a shRNA or siRNA which inhibits expression of UBE2L6, or (iii) a small molecule inhibitor of E2 ubiquitin ligase, and a pharmaceutically acceptable carrier.

Also provided is a method for treating obesity, treating an obesity comorbidity, or treating a dyslipidemia in a subject comprising administering to the subject an amount of an enhancer of UBE2L6 conjugation with ISG15 effective to treat the obesity, obesity comorbidity, or dyslipidemia in the subject.

As used herein, to “treat” obesity in a subject, or a grammatical equivalent thereof, means to stabilize, reduce, ameliorate or eliminate a sign or symptom of the obesity in the subject, or to reduce or prevent further development of obesity in the subject. “Obesity” is generally characterized in the art as the subject having a body mass index of 30.0 or greater (and thus includes the states of significant obesity, morbid obesity, super obesity, and super morbid obesity). In regard to gender, women with over 30% body fat are considered obese, and men with over 25% body fat are considered obese.

The methods of treating obesity as disclosed herein are also applicable, mutalis mutandis, to treating an overweight state in a subject, which is defined as a body mass index of the subject of from 25.0 to 29.9, so as to stabilize, reduce, ameliorate or eliminate a sign or symptom of the overweight state in the subject or to reduce or prevent further development of the subject becoming more overweight.

As used herein, to “treat” an obesity comorbidity in a subject who has an obesity comorbidity, or grammatical equivalent thereof, means to stabilize, reduce, ameliorate or eliminate a sign or symptom of the obesity comorbidity in the subject or to prevent or reduce further development of the obesity comorbidity. Obesity comorbidities include type II diabetes, insulin resistance, coronary heart disease, glucose intolerance, cerebrovascular disease, high blood pressure, gout, gallstones, colon cancer, sleep apnea, and nonalcoholic fatty liver disease (NAFLD).

In an embodiment, the subject being treated is susceptible to obesity. As used herein, a subject who is “susceptible to obesity” means a subject who is likely to develop obesity, or susceptible to worsening an already extant obese state, by way, for example, of diet, environment, drug treatment, or genetic predisposition. As used herein, to treat a subject who is susceptible to obesity means to reduce, attenuate or impair a body mass increase in the subject. As used herein, a subject who is susceptible to an obesity comorbidity means a subject who has obesity, or is developing obesity, and is susceptible to worsening an already extant obesity comorbidity or susceptible to developing the obesity comorbidity, by way, for example, of diet, environment, or genetic predisposition. As used herein, to treat a subject who is susceptible to an obesity comorbidity means to reduce, attenuate or impair development of the obesity comorbidty, or to reduce, attenuate or impair worsening of the obesity comorbidity. In an embodiment of the methods described herein the subject is susceptible to obesity.

In an embodiment, the subject being treated has a dyslipidemia. As used herein, a “dyslipidemia” is an abnormal amount of lipids (e.g. cholesterol and/or fat) in the blood. Dyslipidemia is elevation of plasma cholesterol, triglycerides (TGs), or both, or a low high-density lipoprotein level that contributes to the development of atherosclerosis. Causes may be primary (genetic) or secondary. Diagnosis is by measuring plasma levels of total cholesterol, TGs, and individual lipoproteins. Dyslipidemias are medically-recognized (see The Merck Manual of Diagnosis and Therapy, 18^{th} Edition, Merck Publishing, ISBN-10: 0911910182, the content of which is hereby incorporated by reference). In an embodiment the dyslipidemia results in or is caused by obesity in the subject. In an embodiment the obesity is caused by a high fat diet. In an embodiment the dyslipidemia is excess fatty acid synthesis. In an embodiment the dyslipidemia is excess cholesterol synthesis. To “treat” a dyslipidemia as used herein means to reduce, ameliorate, arrest or reverse one or more symptoms of the dyslipidemia.

In an embodiment, the UBE2L6 is encoded by the gene described in NCBI. Reference Sequence: NC_{—}000011.9.

In an embodiment, ISG15 is Interferon-induced 17 kDa protein that in humans is encoded by the ISG15 gene. In an embodiment, ISG15 is encoded by the gene described in NCBI Reference Sequence: NC_{—}000001.10.

In an embodiment, UBE2L6 (the enzyme) has the sequence:

```
(SEQ ID NO: 1)
                10 20 30 40
MMASMRVVKE LEDLQKKPPP YLRNLSSDDA NVLVWHALLL
                50 60
PDQPPYHLKA FNLRISFPPE
                70 80 90 100
YPFKPPMIKF TTKIYHPNVD ENGQICLPII SSENWKPCTK
              110 120
TCQVLEALNV LVNRPNIREP
              130 140 150
LRMDLADLLT QNPELFRKNA EEFTLRFGVD RPS
```

As used herein, a shRNA (small hairpin RNA) or siRNA (small interfering RNA) directed to a target means an shRNA or siRNA, respectively, effective to inhibit expression of the target. In an embodiment, the siRNA as used in the methods or compositions described herein comprises a portion which is complementary to an mRNA sequence encoded by NCBI Reference Sequence: NC_{—}000011.9, and the siRNA is effective to inhibit expression of UBE2L6. In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. The siRNA can be 5′ phosphorylated or not and may be modified with any of the known modifications in the art to improve efficacy and/or resistance to nuclease degradation. In an embodiment the siRNA can be administered such that it is transfected into one or more cells.

In one embodiment, a siRNA of the invention comprises a double-stranded RNA wherein one strand of the RNA is 80, 85, 90, 95 or 100% complementary to a portion of an RNA transcript of a gene encoding UBE2L6. In another embodiment, a siRNA of the invention comprises a double-stranded RNA wherein one strand of the RNA comprises a portion having a sequence the same as a portion of 18-25 consecutive nucleotides of an RNA transcript of a gene encoding UBE2L6. In yet another embodiment, a siRNA of the invention comprises a double-stranded RNA wherein both strands of RNA are connected by a non-nucleotide linker. Alternately, a siRNA of the invention comprises a double-stranded RNA wherein both strands of RNA are connected by a nucleotide linker, such as a loop or stem loop structure.

In an embodiment, a single strand component of a siRNA of the invention is from 14 to 50 nucleotides in length. In another embodiment, a single strand component of a siRNA of the invention is 14, 15, 16, 17, 18, 19, 20, 21, 22, 23, 24, 25, 26, 27, or 28 nucleotides in length. In yet another embodiment, a single strand component of a siRNA of the invention is 21 nucleotides in length. In yet another embodiment, a single strand component of a siRNA of the invention is 22 nucleotides in length. In yet another embodiment, a single strand component of a siRNA of the invention is 23 nucleotides in length. In one embodiment, a siRNA of the invention is from 28 to 56 nucleotides in length. In another embodiment, a siRNA of the invention is 40, 41, 42, 43, 44, 45, 46, 47, 48, 49, 50, 51, or 52 nucleotides in length. In yet another embodiment, a siRNA of the invention is 46 nucleotides in length.

In an embodiment, an siRNA of the invention comprises at least one 2′-sugar modification. In another embodiment, an siRNA of the invention comprises at least one nucleic acid base modification. In an embodiment, an siRNA of the invention comprises at least one phosphate backbone modification.

As used herein, the term “antibody” refers to complete, intact antibodies. As used herein a “fragnient” of an antibody refers to a Fab, Fab′, F(ab)₂, and other fragments of antibodies which fragments bind the antigen of interest, in this case UBE2L6. Complete, intact antibodies include, but are not limited to, monoclonal antibodies such as murine monoclonal antibodies, polyclonal antibodies, chimeric antibodies, human antibodies, and humanized antibodies. Fragments of antibodies may be fragments of any of these antibodies.

Various forms of antibodies may be produced using standard recombinant DNA techniques (Winter and Milstein, Nature 349: 293-99, 1991). For example, “chimeric” antibodies may be constructed, in which the antigen binding domain from an animal antibody is linked to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobulin light chain or heavy chain) (see, e.g., Cabilly et al., U.S. Pat. No. 4,816,567; Morrison et al., Proc. Natl. Acad. Sci. 81: 6851-55, 1984). Chimeric antibodies reduce the immunogenic responses elicited by animal antibodies when used in human clinical treatments. In addition, recombinant “humanized” antibodies may be synthesized. Humanized antibodies are antibodies initially derived from a nonhuman mammal in which recombinant DNA technology has been used to substitute some or all of the amino acids not required for antigen binding with amino acids from corresponding regions of a human immunoglobulin light or heavy chain. That is, they are chimeras comprising mostly human immunoglobulin sequences into which the regions responsible for specific antigen-binding have been inserted (see, e.g., PCT patent application WO 94/04679). Animals are immunized with the desired antigen, the corresponding antibodies are isolated and the portion of the variable region sequences responsible for specific antigen binding are removed. The animal-derived antigen binding regions are then cloned into the appropriate position of the human antibody genes in which the antigen binding regions have been deleted. Humanized antibodies minimize the use of heterologous (inter-species) sequences in antibodies for use in human therapies, and are less likely to elicit unwanted immune responses. Primatized antibodies can be produced similarly.

Another embodiment of the antibodies employed in the compositions and methods of the invention is a human antibody, which can be produced in nonhuman animals, such as transgenic animals harboring one or more human immunoglobulin transgenes. Such animals may be used as a source for splenocytes for producing hybridomas, as is described in U.S. Pat. No. 5,569,825.

Antibody fragments and univalent antibodies may also be used in the methods and compositions of this invention. Univalent antibodies comprise a heavy chain/light chain dimer bound to the Fc (or stem) region of a second heavy chain. “Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′), as well as tetramers which correspond to the two branch segments of the antibody Y, (commonly known as F(ab)₂), whether any of the above are covalently or non-covalently aggregated, so long as the aggregation is capable of specifically reacting with a particular antigen or antigen family.

The antibody (intact of fragment) can be conjugated to a molecule which permits the antibody to cross the cell membrane or which aids in internalization of the antibody by adipocytes.

As used herein, the term “bind”, or grammatical equivalent, means the physical or chemical interaction between two proteins or compounds or associated proteins or compounds or combinations thereof, including the interaction between an antibody and a protein. Binding includes ionic, non-ionic, hydrogen bonds, Van der Waals, hydrophobic interactions, etc. The physical interaction, the binding, can be either direct or indirect, indirect being through or due to the effects of another protein or compound. Direct binding refers to interactions that do not take place through or due to the effect of another protein or compound but instead are without other substantial chemical intermediates.

Small molecule inhibitors of E2 ubiquitin ligase activity are known (e.g., see Ceccarelli D F ct al., Cell. 145(7):1075-87 (2011)).

The compositions of this invention, or the compounds or compositions as used in the methods of this invention, may be administered in various forms, including those detailed herein. The treatment with the compound may be a component of a combination therapy or an adjunct therapy, i.e. the subject or patient in need of the drug is treated or given another drug for the disease (e.g. a statin for treating dyslipidemia) in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier.

The dosage of the recited compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with anti-lipogenic compounds. The compounds can be administered in oral dosage forms as tablets, capsules, pills, powders, granules, elixirs, tinctures, suspensions, synips, and emulsions. The compounds may also be administered in intravenous (bolus or infusion), intraperitoneal, subcutaneous, or intramuscular form, or introduced directly, e.g. by injection or other methods, into the cancer, all using dosage forms well known to those of ordinary skill in the pharmaceutical arts.

The compounds can be administered in admixture with suitable, pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be coadministered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol. 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol. 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds can also be administered in the form of liposome delivery systems, such as small unilamallar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds may also be coupled to soluble polymers as targetable drug carriers or as a prodrug. Such polymers include polyvinylpyrrolidone, pyran copolymer, polyhydroxylpropylmethacrylamide-phenol, polyhydroxycthylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfate, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field, the content of which is hereby incorporated by reference.

The compounds of the instant invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen. Parenteral and intravenous forms may also include minerals and other materials to make them compatible with the type of injection or delivery system chosen.

In an embodiment of the methods disclosed herein the subject is a human. In an embodiment of the methods disclosed herein the subject is woman. In an embodiment of the methods disclosed herein the subject is a man.

Where a numerical range is provided herein, it is understood that all numerical subsets of that range, and all the individual integers contained therein, are provided as part of the invention. Thus, an siRNA which is from 20 to 25 nucleotides in length includes the subset of siRNA which are 20 to 23 nucleotides in length, the subset of siRNA which are 22 to 24 nucleotides in length etc. as well as an siRNA which is 20 nucleotides in length, an siRNA which is 21 nucleotides in length, an siRNA which is 22 nucleotides in length, etc. up to and including an siRNA which is 25 nucleotides in length.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

The previously-reported low body fat content of leptin deficient BALB/c mice (2) has been utilized herein to develop an understanding of leptin-independent regulation of adipocyte triglyceride storage. Comparisons of the metabolic parameters between leptin deficient BALB/c, C57BL/6J and F1 hybrids have indicated that a difference in fatty acid oxidation rates, driven by basal lipolysis rates, is the major metabolic parameter that is associated with body fat content in these mouse models. It is stated herein that basal lipolysis rate is a significant factor in regulating triglyceride stores in adipocytes. Molecular studies of adipocyte lipid metabolism and a genome wide scan of backcrossed N2 ob/ob progeny indicate that the regulation of adipocyte concentrations of adipocyte triglyceride lipase (ATGL) is likely to be major factor in controlling basal lipolysis rates in adipocytes (3) as the leaner BALB/c mice have higher concentrations of ATGL. Turnover rates of ATGL appear to be impacted by the ubiquitination/ISG15 pathway. Genetic association studies show that the gene coding for a catalytic subunit of the ubiquitination enzyme complex, Ubc216 (Ubch8), shows coding sequence variation between the BALB/c and C57BL/6J strains. Interestingly, UBE2L6 (4, 5) is an E2 enzyme that functions in the ubiqutination process (6, 7) that can also conjugate with ISG15, a ubiquitin like peptide, to a wide variety of protein substrates (8). It is possible that the amino acid variants for Ube216 affect its ability to ubiquitinate its substrates and that turnover rates of ATGL are consequently impacted by the ubiquitination/ISG15 process.

Impact on Body Fat Content of Overexpression of ATGL within Adipocytes of ob/ob mice.

Data disclosed herein show that ATGL is significantly reduced in B6 ob/ob mice relative to the leaner BALB/c ob/ob mice. Reduced lipolysis rates, due to lowered ATGL, can thus be a bottleneck in lipid metabolism and increasing ATGL will increase basal lipolysis and fatty acid oxidation with a subsequent reduction in adipocyte triglyceride content.

Correlation of the Impact of Ube216 Genetic Variants on the Body Fat Content and ATGL Concentrations in BALB/c Ob/Ob Mice.

Data disclosed herein indicate that the BALB/c variant of UBE2L6 is likely to be highly conjugated with ISG15 whereas the C57BL/6J variant is much less ISG15ylated, if at all. A higher degree of ISG15ylation likely contributes to the longer lifetime and higher concentration of ATGL in BALB/c mice. The C57BL/6J allele of Ube216 introduced into the BALB/c strain via backcrossing will likely lower the degree of ISG15ylation of UBE2L6 and decrease the amount of ATGL in BALB/c mice, thereby decreasing basal lipolysis rates and increasing adipocyte triglyceride content.

Role of ISG15 in the Control of ATGL Degradation.

Data disclosed herein indicate that ISG15ylation of UBE2L6 is a prominent characteristic of the difference between the C57BL/6J and BALB/c strains. Introducing the Isg15 null allele into the BALB/c strain and determining whether ATGL concentrations in adipocytes is altered will clarify if Ube216 amino acid variants are directly responsible for ubiquitinating activity. Both possibilities can be tested by ablating Isg15 expression.

It is proposed herein that in cases of established obesity and associated leptin resistance, adipose tissue has been re-modeled to retain triglycerides due to increased lipogenesis (12). Data disclosed herein suggest increasing basal lipolysis within adipocytes will release fatty acids to the circulation and increase fatty acid oxidation, countering the increase in lipogenesis. Circulating fatty acids are estimated to constitute about 40-50% of circulating fuels under basal conditions in lean individuals (13) with a relative increase during moderately strenuous exercise. Thus, a diminished rate of basal lipolysis within adipocytes, in cases of obesity and leptin resistance, acts as a bottleneck to the reduction of triglyceride stores. Indeed, leptin deficiency is one of the first states that was initially reported to show diminished ATGL expression (14). Moreover, increasing fatty acid oxidation will spare and preserve lean mass.

Lcptin Insensitivity: LIPOGENESIS→HIGH TRIGLYCERIDE STORES→Lipolysis

Leptin Insensitivity+Increased Basal Lipolysis LIPOGENESIS→Normalized triglyceride stores→LIPOLYSIS

Increasing lipolysis and fatty acid oxidation is distinctly different from modalities that restrict adipocyte differentiation (15-17) (leading to metabolic abnormalities akin to lipodystrophy), promote brown fat hypertrophy (18) or brown fat-like properties within white adipocytes (19) (which may involve irreversibly affecting cell fate and determination with attendant concerns of oncogenesis).

Herein a molecular target is disclosed, adipocyte triglyceride lipase (ATGL), as well as a regulatory mechanism, ubiquitination, and ISG15ylation, that can be manipulated to increase basal lipolysis rates. While ATGL has been previously shown to alter adipose triglyceride content in overexpression (20) and knockout (21, 22) models, evidence is provided for a mechanism that regulates concentrations of ATGL within adipocytes by modulating protein turnover rate independently of transcription. While deficiencies in ATGL or CGI-58 in mice cause an obesity phenotype (21), humans exhibit an epidermal phenotype due to neutral lipid storage disease (Chanarin-Dorfman Syndrome) (22). However, increased ATGL activity in mice and humans may achieve the same target of increasing fatty acid availability for oxidation and reducing body fat content.

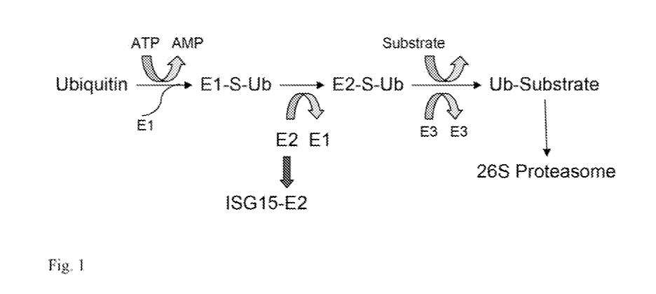

The data herein is consistent with a major target for ISG15ylation within fat cells being UBE2L6, an E2 subunit of an ubiquitination enzyme complex (5). Typically, ubiquitination requires three proteins: E1 to activate the ubiquitin, E2 to catalyze the ubiquitinaton of the substrate and E3 for recognition of the specific protein substrate (see FIG. 1). Ubiquitin is the prototype of a family of ubiquitin like peptides that are conjugated to various proteins for targeted degradation by the 26S proteasome (23). Ubiquitin like peptides use a similar three protein complex to achieve substrate conjugation and most E2 subunits are believed to be specific for the ubiquitin like peptide that they utilize for catalysis. However, it has recently been shown that UBE2L6 can use both ubiquitin and ISG15 for substrate conjugation (5). The data indicate that ISG15ylation of UBE2L6 is associated with higher accumulation of ATGL without increased transcription rates. The ISG15ylation of UBE2L6 withdraws UBE2L6 from the pool of proteins that can be transacylated with ubiquitin, effectively reducing the ability of the adipocyte to ubiquitinate and degrade ATGL.

A genetic variant in the coding sequence of UBE2L6 appears to be responsible for the rate of ISG15ylation of UBE2L6. The two allelic variants found herein are distributed among some of the oldest inbred mouse strains developed for research (C57BL/6, DBA and BALB/c), suggesting that these genetic variants were prevalent prior to the establishment of inbred mouse lines and may have functional significance.

The BALB/c Ob/Ob Model—a Leptin Deficient Mouse with Relatively Reduced Body Fat Content.

The BALB/c ob/ob mouse model, initially reported upon by Dr. Farid Chehab's group, was identified as having a lower body fat content than the prototypical C57BL/6J ob/ob mouse (2). A congenic strain of BALB/cJ ob was developed by backcrossing (to N5) the Lep-ob mutation from C57BL/6J. In addition, the Agrp KO allele was included in the backcross due to interest in the contribution of AGRP to reproduction and puberty. The data are based on BALB/c ob/b Agrp−/− compared to C57BL/6J ob/ob Agrp−/− (B6). For the purpose of clarity in subsequent discussion, the Agrp−/− designation has been skipped for brevity as no significant differences between the two mouse models due to Agrp ablation were observed.

TABLE 1
Metabolic characteristics of ob/ob female mice on BALB/c
and C57BL/6 backgrounds, including F1 hybrids.

| | | BALB/c ob/ob | C57BL6/J ob/ob | BXC F1 ob/ob |
|---|---|---|---|---|
| Body Weight (g) | | 37.8 ± 1.1 * | 47.4 ± 1.6 | 50.6 ± 1.0 |
| Fat mass (g) | | 17.3 ± 0.8 * | 28.4 ± 1.25 | 30.3 ± 0.8 |
| Liver mass (g) | | 2.31 ± 0.2 | 2.39 ± 0.1 | 2.7 ± 0.24 |
| Food intake (g) | | 7.13 ± 0.6 * | 10.9 ± 0.2 | 7.71 ± 0.8 * |
| EE-VO2 | Day | 4538 ± 157 | 4520 ± 696 | 4842 ± 467 |
| (ml/kg/hr) | Night | 4629 ± 145 | 4639 ± 998 | 4582 ± 436 |
| | Total | 4590 ± 148 | 4585 ± 855 | 4692 ± 442 |

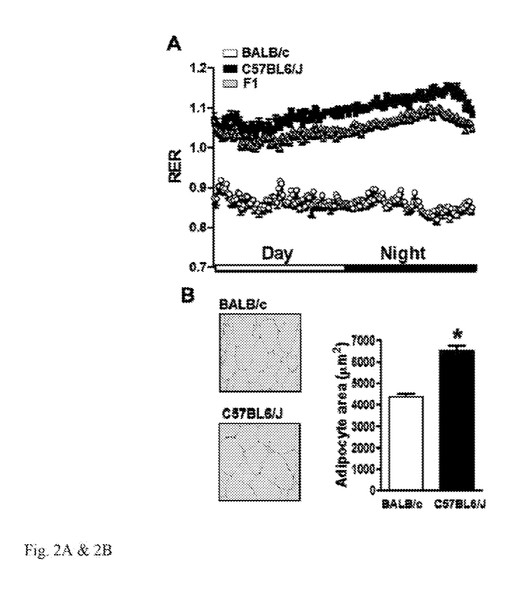

Table 1 shows body composition and metabolic characteristics of ob/ob mice on two strain backgrounds. Data were collected from 8-10 mice per strain at 3 months of age. Fat mass was determined by magnetic nuclear resonance. Food intake was measured simultaneously with energy expenditure (EE). VO₂ is normalized to fat free mass per mouse. An asterisk designates a difference (p<0.05) from the C57BL/6 ob/ob group, in pairwise analyses. Data from BALB/c ob/ob, C57BL/6J ob/ob and F1 (BALB/c×C57BL/6J) ob/ob mice is presented. Body composition analyses indicate that BALB/c ob/ob mice have significantly less body fat (˜10 g for females and ˜15 grams for males) with little alteration to fat free mass, relative to the B6 and F1 ob/ob mice. Energy balance studies indicate that BALB/c ob/ob mice eat much less than C57BL/6J ob/ob mice but no more than F1 B×C ob/ob mice. As the F1 ob/ob mice also are relatively normophagic while having higher body fat content, the normophagia can be excluded as a major factor in the lower fat content of BALB/c ob/ob mice. Indirect calorimetry also indicated that oxygen consumption, normalized to lean body mass, did not differ between the strains. However, BALB/c ob/ob mice have near normal respiratory exchange ratios (RER) of ˜0.8 (FIG. 2) whereas B6 and F1 ob/ob animals have ratios close to 1.0 or above. This difference in RER indicates a difference in substrate utilization wherein the BALB/c ob/ob mice oxidize a mix of carbohydrates and fatty acids whereas B6 and F1 ob/ob mice barely oxidize fatty acids, relying primarily upon carbohydrates and amino acids. Indeed, B6 ob/ob mice have RERs that are consistently above 1.0, indicating the chronic persistence of de novo lipogenesis, contributing to excess triglyceride retention. Histological examination of fat from the two lines indicate that BALB/c ob/ob animals have smaller adipocytes that B6 ob/ob mice, (FIG. 2). It is concluded that increased fatty acid oxidation in the BALB/c ob/ob mice was responsible for their lower body fat content. Furthermore, the non-adipose tissue is responsible for the increased rates of fatty acid oxidation as no differences in expression of fatty acid oxidative enzyme RNAs or UCPs were observed in the WAT of BALB/c and B6 ob/ob mice (data not shown).

Increased Basal Lipolysis in BALB/c ob/ob Mice is a Cell Autonomous Function of BALB/c Adipocytes.

To obtain further information regarding lipolysis, circulating concentrations of glycerol were measured as a proxy for lipolysis rates. Table 2 indicates that BALB/c ob/ob mice have higher glycerol concentrations that B6 and F 1 ob/ob mice, indicating higher basal lipolysis rates. This would suggest the presence of facultative rates of fatty acid oxidation, at least within the BALB/c ob/ob mice. Interestingly, BALB/c ob/ob mice have lower glucose concentrations in their blood along with an improved glucose tolerance test, indicative of improved glucose tolerance/insulin sensitivity, which would tend to promote lipogenesis. Indeed, higher amounts of acetyl-CoA carboxylase and phospho-ACC (Ser79) in BALB/c ob/ob fat were observed relative to B6 ob/ob fat, indicating that lipogenesis in BALB/c ob/ob mice is not reduced (data not shown). Thus, it is unlikely that differences in lipogenesis contribute to the disparity in fat content between the strains.

TABLE 2
Higher rates of basal lipolysis in ob/ob BALB/c mice despite improved
insulin sensitivity.

| | | BALB/c ob/ob | | C57BL6/J ob/ob | | F1 ob/ob | |
|---|---|---|---|---|---|---|---|
| | | ♂ | ♀ | ♂ | ♀ | ♂ | ♀ |
| Glycerol | Fed | | 58.7 ± 2.4 | | 49.9 ± 1.8 * | | 49.9 ± 2.5 * |
| (mg/dl) | Fasted | | 53.6 ± 3.0 | | 52.2 ± 1.2 | | nd |
| NEFA | Fed | 1149 ± 69.4 | 832.6 ± 118 | 689.8 ± 47.6 * | 902.3 ± 102 | | |
| (μM) | Fasted | 1614 ± 121 | 1450 ± 106 | 1100 ± 103 * | 1122.6 ± 180 | | |
| Glucose (mg/dl) | Fasted | 151.2 ± 22.6 | 91.7 ± 7.6 | 226.2 ± 28.4 * | 170.8 ± 37.6 * | 308.5 ± 46.8 * | 161.8 ± 32.3 * |
| Insulin (ng/ml) | Fasted | 16.81 ± 2.9 | 17.44 ± 2.2 | 14.24 ± 5.43 | 13.36 ± 3.3 | 8.18 ± 2.6 * | 6.14 ± 0.75 * |

| | | | |
|---|---|---|---|
| GTT (AUC) | 671.2 ± 107.1 | 1890.3 ± 247.7 * | nd |

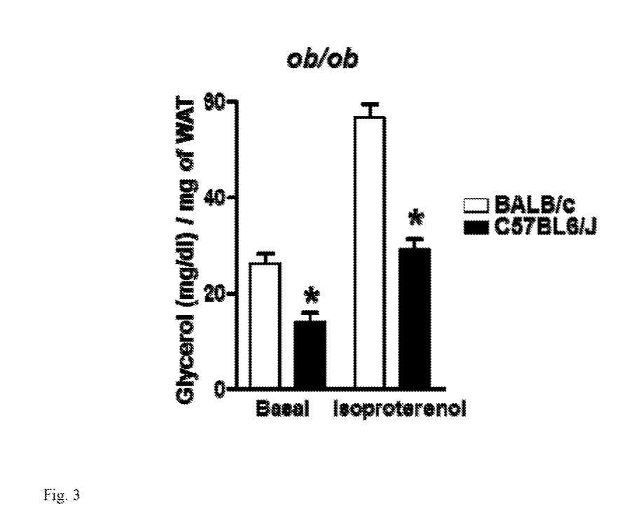

Table 2 shows circulating glycerol and fatty acid concentrations were measured in the fed and fasted states of BALB/c, B6 and F1 ob/ob mice. Glucose and insulin concentrations were also determined in the fasted state. BALB/c ob/ob mice showed improved glucose tolerance during a glucose tolerance test. 5-6 mice were used per genotype group. An asterisk denotes a significant difference (p<0.05) from the sex-matched BALB/c ob/ob group. Fat pad fragments were prepared for examining rates of lipolysis independent of the influences of innervation. These incubations, with and without adrenergic stimulation, indicated that basal and isoproterenol-stimulated rates of lipolysis were elevated in isolated fat fragments of BALB/c ob/ob mice, relative to B6 ob/ob fat fragments (FIG. 3), indicating that the trait is a cell autonomous function.

Increased Amounts of Adipose Triglyceride Lipase (ATGL) in BALB/c Ob/Ob Adipocytes.

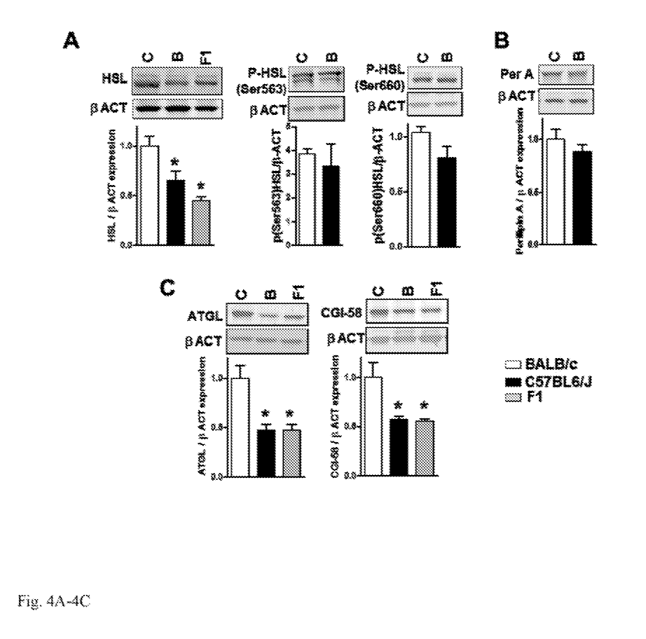

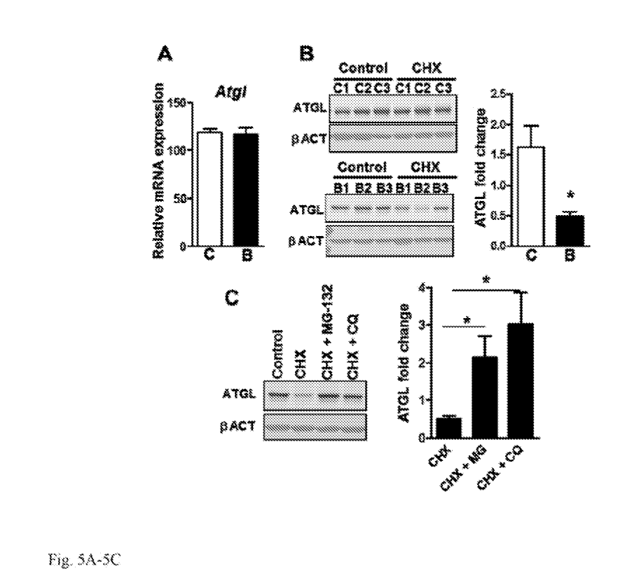

Lipolysis in rodents and humans is primarily regulated by adipose triglyceride lipase (ATGL) and hormone sensitive lipase (HSL) (24). Increasing expression of either lipase (HSL and ATGL) in the liver releases fatty acids from hepatocytes and reduces hepatosteatosis (25). Herein the contributions of ATGL and HSL to basal lipolysis, as well as perilipin, another putative regulator of lipolysis, are examined. Examination of transcript concentrations by quantitative RT-PCR did not yield any differences between the two strains (FIG. 5 for ATGL mRNA quantification). Examination of protein concentrations of ATGL and HSL with Western blots (FIG. 4) showed that there are increased amounts of both ATGL and HSL in BALB/c ob/ob fat, relative to B6 ob/ob fat. We did not see differences in Perilipin A protein. Amounts of phosphorylated HSL, the activated form of HSL, were examined and it was found that there were no differences in the amounts of phospho-HSL (Ser563 and Ser660) between the two mouse strains. The co-lipase for ATGL, CG158, was also examined and higher concentrations of CG158 in BALB/c ob/ob fat (FIG. 4) were found compared to B6 and F1 ob/ob fat.

Fat fragments incubated with cycloheximide, an inhibitor of protein synthesis, indicated that fat from B6 ob/ob mice had a dramatic reduction in ATGL concentrations whereas BALB/c ob/ob fat showed no alteration in ATGL amounts after cycloheximide treatment (FIG. 5). Thus, BALB/c ob/ob fat has long lived ATGL whereas C57BL/6 ob/ob fat has ATGL that is degraded within several hours. When B6 ob/ob fat, was co-treated with cycloheximide and MG-132, a proteasomc inhibitor, or chloroquine, a lysosome inhibitor, ATGL concentrations persisted (FIG. 5) in a manner similar to BALB/c ob/ob fat. Thus, ATGL appears to be degraded by both the lysosomal and proteasomal pathways.

Free fatty acids (FFA) release from adipose tissue explants was further measured in presence or not of the HSL inhibitor CAY 10499. The efficiency of CAY 10499 in our experimental conditions had been previously assessed—it was found that induced lipolysis with a selective β3AR agonist (CL316, 243) is blunted in presence of 100 μM CAY10499. FFA release was compared from freshly dissected adipose tissue explants from BALB/c or B6 ob/ob inguinal fat pad. It was observed that FFA production was increased in explants from BALB/c relative to B6 ob/ob mice when HSL activity is inhibited by CAY10499. The same difference between BALB and B6 lipolysis (Δ^{-CAY}=43 10.3±31.7 versus Δ^{+CAY}=397.1±8.1, P=0.31) without the HSL inhibitor (i.e. HSL+ATGL activity) or with CAY10499 (i.e. ATGL activity), respectively. Consequently, the data show that increased adipose lipolytic rate in BALB/c principally relied on increased ATGL activity. Of note, the increased ATGL activity is greater than the increase in ATGL protein content in adipose tissue. This may be due to the concomitant increase of CGI-58 expression that is known to enhance ATGL lipolytic activity.

A Genome Wide Scan Points to Loci on Chromosomes 2 and 3 Associated with Body Fat Fraction.

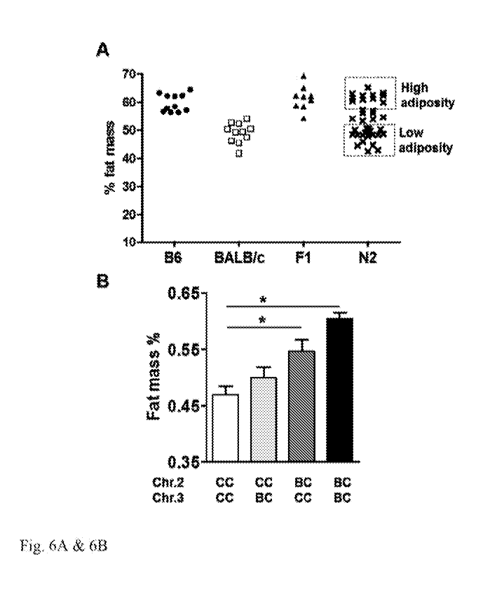

Using a complementary approach, a genome wide scan was performed in ob/ob mice from two cohorts (45-50 ob/ob mice per cohort) of an N2 backcross (F1×BALB/c). The N2 progeny had a wide spectrum of body fat fraction (FIG. 6) and the obese N2 progeny (ob/ob mice verified by genotype) were examined within the low and high extremes of fat content with a two-cohort strategy for replication. Markers of Chromosomes 2 and 3 were associated with body fat fraction (Table 3), replicated by the second cohort (26). Analysis of each QTL suggests that the Chromosome 2 locus has a larger effect although the two QTLs appear to be additive and are sufficient to explain the differences between the parental strains of ˜15% body fat fraction (FIG. 6). The Chr 2 locus was focused on after data mining had identified Ube216, an E2 subunit of the ubiquitination complex, that is located ˜10 Mbp away from D2Mit37, the Chr 2 marker at 74.5 Mbp with the highest level of significance in the genome scan. It is believed that Ube216 is the gene that is the Chr 2 locus controlling body fat content variation between B6 and BALB/c ob/ob mice.

TABLE 3
Association of Chr 2 and Chr 3 markers with body fat content in ob/ob
N2 (F1 × F1) backcross progeny.

| | | Set #1 | | | Set #2 | | | |
|---|---|---|---|---|---|---|---|---|
| | | High | Low | | High | Low | | Set #1 + |
| | | (fat | (fat mass | | (fat | (fat | | #2 |
| | | mass %) | %) | P | mass %) | mass %) | P | P |
| Mean fat mass % | | 61.8 ± 0.62 | 47.7 ± 0.79 | <0.0001 | 60.9 ± 0.64 | 44.7 ± 1.3 | <0.0001 | <0.0001 |
| | Pos. | | | | | | | |
| Markers | (Mb) | CC:BC genotype ratios | | | | | | |
| rs 3678168 | 5.57 | 2:7 | 6:7 | 0.25 | | | | |
| D2Mit37 | 74.5 | 0:9 | 9:4 | 0.001 | 3:13 | 9:5 | 0.011 | <0.0001 |
| D2Mit42 | 104 | 1:8 | 8:5 | 0.018 | 5:12 | 8:6 | 0.11 | 0.010 |
| D2Mit30 | 124.7 | 3:6 | 7:6 | 0.25 | 7:10 | 7:7 | 0.62 | 0.26 |
| D2Mit456 | 168.7 | 4:5 | 6:7 | 0.89 | nd | nd | | |
| D3Mit117 | 5.8 | 2:7 | 9:4 | 0.03 | 7:10 | 9:5 | 0.2 | 0.0196 |
| D3Mit64 | 49.9 | 3:6 | 10:3 | 0.04 | 7:10 | 9:5 | 0.2 | 0.0196 |
| D3Mit230 | 82.3 | 3:6 | 9:4 | 0.096 | nd | nd | | |
| D3Mit29 | 90.7 | 3:6 | 9:4 | 0.096 | 7:10 | 8:6 | 0.37 | 0.0745 |

Table 3 shows markers were scored by agarose gel electrophoresis of DNA fragments amplified by PCR. Allele distributions were analyzed by chi square frequency analysis, assuming random distributions for the null hypothesis. No correction for multiple testing was used but results were replicated with mice in Set 2, an independent panel of ob/ob N2 progeny.

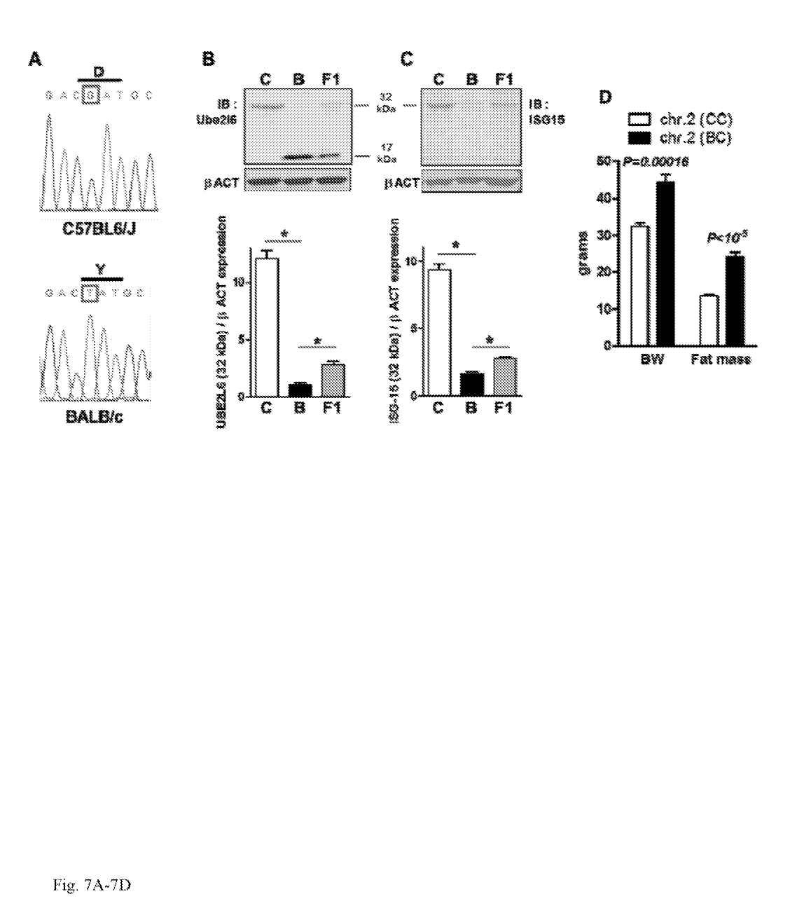

The public database indicated the potential presence of 3 coding sequence differences in Ube216 between C57BL/6J and BALB/c. Sequence analysis was performed with genomic DNA from C57BL/6J and BALB/cJ mice from our colonies as well as mice directly obtained from JAX. One sequence variant was verified that results in an amino acid sequence difference (FIG. 7). The two other reported allelic variants were not identified. A subcongcnic strain of BALB/c ob/ob Agrp−/− carrying a segment of C57BL/6 Chr 2, including Ube216 was developed. Initial body composition analyses of these mice indicate that the B6 Chr 2 segment causes increase in fat content, relative to age matched BALB/c ob/ob Agrp−/− mice (FIG. 7).

Strain Differences in Post-Translational Modifications of Ube216.

UBE2L6 was examined in the fat pads of BALB/c ob/ob and B6 ob/ob mice and a dramatic difference found in the electrophoretic migration of the UBE2L6 protein between the two strains. While B6 ob/ob fat showed the expected 17 kDa band for UBE2L6, the majority of the UBE2L6 signal from BALB/c ob/ob fat migrated at 32 kDa (FIG. 7). Fat pads from ob/ob F1 mice contained bands for both the 17 and 32 kDa bands that were of lower intensities for the respective bands in the WAT of the two parental strains, consistent with a gene dosage effect. As the samples were run under reducing conditions, ester bonds between UBE2L6 and ubiqutin or ubiquitin like peptides formed by the transfer of these peptides from the E1 component would have been eliminated. This suggested that the modification is not the result of the typical cycling of ubiquitin like peptides between subunits of the ubiquitination complex.

Adipocyte protein extracts were probed with antisera to ISG15 and ubiquitin. The expected smear was not observed with a ubiquitin antibody blot of anti-UBE2L6 immunoprecipitates from protein extracts of either strain. However, a strong signal (FIG. 7) was readily observed at 32 kDa from adipocyte protein extracts of BALB/c ob/ob mice which is nearly absent from B6 ob/ob fat extracts. Extracts from F1 ob/ob mice had a signal at 32 kDa that was intermediate in intensity to B6 and BALB/c bands, consistent again with a gene dosage effect. Thus, the coding sequence BALB/c variant for Ube216 probably leads to reduced proteolysis rates of ATGL.

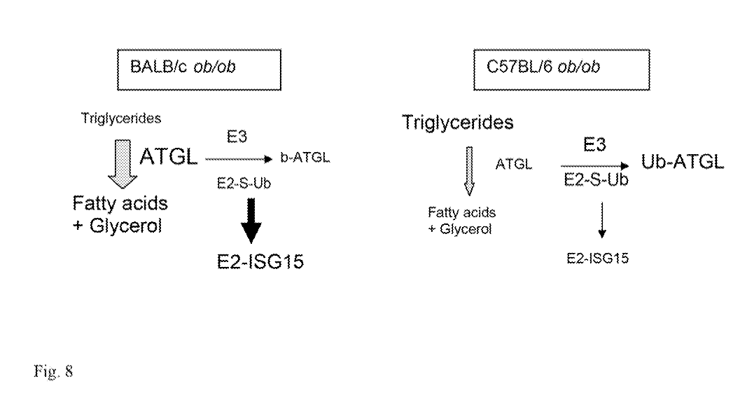

Thus, a comprehensive model has been developed that can provide a molecular mechanism for the strain differences in fat content between ob/ob mice of the BALB/c and C57BL/6 strains (FIG. 8). In BALB/c ob/ob mice, their white adipocytes have a higher rate of fatty acid release due to high steady state concentrations of ATGL. These ATGL concentrations are maintained due to low rates of ubiquitination and subsequent degradation of ATGL. In the adipocytes of C57BL/6 ob/ob mice, low rates of lipolysis lead to accumulation of triglycerides as a consequence of low concentrations of ATGL. High rates of ubiquitination of ATGL lead to rapid degradation of ATGL.

Characterizing the impact on body fat content of overexpression of ATGL within adipocytes of ob/ob mice.

An adipocyte-specific overexpression system for driving ATGL in C57BL/6J ob/ob mice, similar to a previously described model but with an option for inducibility can be produced (20). The data show that ATGL is significantly reduced in B6 ob/ob mice relative to the leaner BALB/c ob/ob mice. It is hypothesized that reduced lipolysis rates, due to lowered ATGL, are a bottleneck in lipid metabolism and increasing ATGL will increase basal lipolysis and fatty acid oxidation with a subsequent reduction in adipocyte triglyceride content.

A CRE-activatable transgene has been constructed and knocked into the Rosa26 locus (27-29), that will express Atgl:Rosa26 promoter-loxP-neo-loxP-ATGL. Several (at least 6) targeted clones have been identified after electroporation into ES cells derived from C57BL/6 mice (30). These clones have been submitted to the Einstein Gene Targeting Core. The initial injection attempt did not yield any chimeras, based on coat color. It was anticipated that some mice would be chimeric for black and white fur as the host blastocysts are from the standard black B6 strain whereas the ES cells are derived from albino C57BL/6 mice. These mice are used for overexpression of ATGL within adipocytes by combining an adipocyte specific CRE transgene with the conditional ATGL transgene. The adiponectin-CRE transgene (31) is initially tested as the transgene. Mice are bred with the following genotypes:
1. adiponectin-CRE Rosa26-lox-ATGL ob/ob (experimental group)
2. adiponectin-CRE ob/ob (control group)
3. Rosa26-lox-ATGL ob/ob (control group)
4. ob/ob

Adiponectin CRE has been denoted as a proxy for either the adiponectin CRE or the dual transgene model adiponectin-rtTA+tetO-CRE. Group 1 is compared to the three other groups (Groups 2, 3 and 4) for body composition and energy balance parameters. Groups 2-4 can be combined for statistical analyses if necessary. Data is obtained regarding body mass, body composition, food intake, energy expenditure and respiratory exchange ratio (via indirect calorimetry). Circulating concentrations of glycerol and fatty acids are determined, along with glucose and insulin concentrations to ascertain any perturbations of insulin sensitivity and glucose handling. Studies are performed on between 6-8 mice of each genotype and both males and females for study.

In the case of the inducible CRE model, the option of comparing mice with and without CRE induction by doxycycline is available. Whole animal studies can include body weight, body composition analysis by NMR, food intake, indirect calorimetry and RER determinations (as done in FIG. 1) with measurements of circulating glycerol and fatty acids (as in Table 2). White adipose tissue is examined for expression of ATGL and CGI58 by mRNA and protein analyses (as in FIGS. 4 and 5). ATGL turnover rates are compared between isolated WAT fragments of the two congenic strains, with addition of cycloheximide. Adipocyte size is ascertained by morphometry of histological specimens. The techniques for these methods have been previously published (25, 33, 34).

Over-expression of ATGL within white adipocytes will increase lipolysis rates and release of fatty acids from adipocytes and reduce the fat stores of the ob/ob mice with the adiponectin-CRE Rosa26-lox-ATGL transgenes. The increased availability of circulating fatty acids will lead to increased whole body fatty acid oxidation as reflected by a lower RER. This will confirm that ATGL regulates lipolysis rates in adipocytes of leptin deficient mice.

Where chimerism of transgene expression (either the CRE or the ATGL transgenes), or insufficient expression of the ATGL transgene or lack of increased ATGL expression by rapid proteolysis targeted toward ATGL within B6 adipocytes causes issues an alternative strategy of ablating Atgl expression in BALB/c ob/ob mice is pursued. Using Atgl KO mice to generate BALB/c ob/ob Atgl-null mice with 5 backcrosses to the BALB/c strain, BALB/c ob/ob are compared to BALB/c ob/ob Atgl-null mice, with the prediction that the loss of Atgl will reduce WAT lipolysis and promote lipid accumulation within white adipose tissue. Atgl KO mice and wild type mice are studied to provide a baseline by which the Atgl KO increases body fat content. While Atgl-null mice have increased fat mass (21) and ATGL transgenic mice have reduced body fat (20), those studies did not examine the effects of manipulating leptin-deficient mice. This alternative strategy will provide evidence supporting the importance of WAT lipolysis in regulating adipocyte lipid stores. The alternative strategy also has the advantage of a uniform ablation of ATGL expression although this effectively alters the baseline body fat content as the Atgl KO mice are mildly obese.

An issue with the treatment modality is the inability of peripheral tissues to oxidize the increased amounts of fatty acids from adipocytes. However, the increase in circulating fatty acids in BALB/c ob/ob mice is disproportionately smaller than the increase in lipolysis (compared to B6 ob/ob parameters), indicating that peripheral tissues are facultative in their use of substrates as fuels. Moreover, the livers of BALB/c ob/ob mice are of a similar weight to B6 ob/ob mice, indicating a similar degree of hepatosteatosis despite the disadvantage of increased lipogenic potential from improved insulin sensitivity in BALB/c ob/ob mice.

Correlating the impact of Ube216 genetic variants on the body fat content and ATGL concentrations in BALB/c ob/ob mice.

The data indicate that the BALB/c variant of UBE2L6 is likely to be highly conjugated with ISG15 whereas the C57BL/6J variant is much less ISG15ylated, if at all (FIG. 7). A higher degree of ISG15ylation contributes to the longer lifetime and higher concentration of ATGL in BALB/c mice. Introduction, via backcrossing, of the C57BL/6J allele of. Ube216 into the BALB/c strain will illuminate this. In the data presented initial body compositions of N5 backcross ob/ob BALB/c mice with one B6 Ube216 allele were compared to two BALB Ube216 alleles. This result is expected as the data indicates that the B6 Ube216 allele is dominant. Further characterizing the metabolic characteristics of these mice along with molecular studies of their adipose tissues permits understanding of the underlying mechanisms.

Introduction of the C57BL/6 Ube216 allele into BALB/c ob/ob mice will lower the degree of ISG15ylation of UBE2L6 and decrease the amount of ATGL in the adipocytes of BALB/c ob/ob mice. This was a phenotype observed in the F1 ob/ob animals and it is likely that this phenotype will be replicated in N5 BALB/e ob/ob Chr 2-B6 congenic line. Thus, energy balance in these mice can be studied by measuring body mass, body composition by magnetic resonance spectroscopy, food intake and energy expenditure by indirect calorimetry in a Columbus Instruments setup. Oxygen consumption, carbon dioxide production and respiratory exchange ratios can be determined over a 4-5 day recording period after an acclimation period of 4-5 days within the calorimeter. Blood can be collected for measurements of glucose, insulin, fatty acids and glycerol. Adipocyte size distributions can be determined from histological speciments of white adipose tissue. Lipolysis of isolate fat fragments can be measured as described in Preliminary Data (FIG. 3). Both sexes can be analyzed with 5-8 mice in each sex and genotype group. (For methods see 25, 33, 34). Measures of RER, a unit free ratio of oxygen consumption and carbon dioxide production, are relied on as a variable that is not linked to corrections for estimation of energy expenditure based on body composition data (35).

The mice are two genotype groups, littermates generated by matings between a BALB/c ob/+ Chr 2 BALB/BALB and a BALB/c ob/+ Chr 2 B6/BALB pairs. These pairings generate BALB/c ob/ob Chr 2 BALB/BALB and BALB/c ob/ob Chr 2 B6/BALB mice, permitting direct comparisons to evaluate the effect of carrying the B6 Chr 2 genomic region carrying Ube216. This breeding strategy randomizes the effects of any unmarked genomic segments derived from the B6 parental strain, an important consideration as the lines are only at the N5 generation and harbor an undetermined amount of the B6 genome. It has previously been estimated that, by the N5 and N6 generations, the amount of the host genome that is unmarked is equivalent in length to the host genome swept along by the selected markers, ˜50-60 cM (26). If the differences are small but significant, mice that are homozygous for the BALB/c and B6 Chr 2 regions can also be generated to enhance detection of differences in physiological and molecular phenotypes.

The B6-derived Chr 2 genomic segment carrying Ube216 will cause increased ubiquitination of ATGL, leading to increased proteolysis of ATGL, lower amounts of steady state ATGL within adipocytes and reduced lipolysis rates. In turn, white adipocytes in BALB/c ob/ob mice carrying the B6 allele of Ube216 will be more obese and have larger adipose stores than BALB/c ob/ob mice. A decrease in whole body fatty acid oxidation rates in BALB/c ob/ob Chr 2 B6/BALB mice will occur which will be reflected in a higher RER. This will show UBE2L6 regulates the rate of degradation of ATGL, thereby regulating lipolysis rates within adipocytes.

To directly address the molecular link between ATGL and UBE2L6, a method has been developed to observe ubiquitination of ATGL in adipocyte extracts from C57BL/6 Mice supplemented with ATP and ubiquitin. Crude lysate from C57BL6/J ob/ob adipose tissue was used as the source for the ubiquitination proteins (E1, E2 and E3). Conjugation of ubiquitin was carried out in a total volume of 50 μl. Reaction mixture contained 250 μg B6 WAT lysate±10 μg of purified ubiquitin, 5 μl of ubiquitinylation buffer (10×) (Enzo Life Sciences), 20 U/ml inorganic pyrophosphate, 1 mM DTT, 5 μl ATP regenerating solution (10×) (Enzo Life Sciences) in presence of MG-132 (proteasome inhibitor) and ubiquitin aldehyde (ubiquitin hydrolase inhibitor) and was incubated at 37° C. for 1 hour. The reaction was stopped with 6×SDS sample bufferer and the products of the reaction were analyzed by SDS-PAGE and visualized after transfer with infrared reagents using the LI-COR system. The assay appears to be a reasonable method of assessing ubiquitination of ATGL in adipocyte extracts. This method is being extended to immunoneutralize UBE2L6 in the extracts to determine the role of UBE2L6 in the ubiquitination process of ATGL. With immunoneutralization of UBE2L6 (either by simple addition of anti-UBE2L6 or coupled to immunoprecipitation with protein A agarose), ubiquitination of ATGL will be significantly reduced. shRNA vectors can also be used for reducing UBE2L6 mRNA and tested for their ability to reduce ATGL. Ube216 is an ideal candidate gene:

1. The body fat content difference between BALB/c and B6 ob/ob mice is correlated with an adipocyte specific function-lipolysis rates.
2. Differences in lipolysis rates and adipocyte sizes between BALB/c and B6 ob/ob adipocytes are highly correlated with steady state concentrations of ATGL, the primary regulator of lipolysis in adipocytes.
3. Ube216 is only ˜10 Mbp distant from the marker with the strongest linkage to body fat content variation in the N2 progeny.
4. The Ube216 coding sequence contains sequence variants between C57BL/6 and BALB/c alleles.
5. Ube216 can be strongly implicated in the ubiquitination mediated degradation of ATGL.
6. Congenic strains of BALB/c ob/ob mice carrying allelic variants of Ube216 show significant variations in body fat composition.

Role of ISG15 in the control of ATGL degradation.

The data indicate that ISG15ylation of UBE2L6 is a prominent characteristic of the difference between the C57BL/6J and BALB/c strains. Introducing the Isg15 null allele (37) into the BALB/c strain and determining whether ATGL concentration in adipocytes of BALB/c ob/ob mice is altered clarifies the various roles. If Ubc216 variants have differences in their ability to be conjugated with ISG15 this results in differences in degree of ubiquitination and concomitant differences in ubiquitinating capacity. Also, if Ube216 BALB/c variant is defective in its ability to transfer ubiquitin to its cognate E3 ligase, independent of its conjugation state with ISG15, and the addition of the Isg15 null allele would have no impact on ATGL concentration or body fat content in BALB/c ob/ob mice.

The Isg15 null allele (up to N5 or N6) is backcrossed to the BALB/c ob line. This is a straightforward process which can be readily monitored with genetic markers for the Isg15 knockout and wild type alleles. The matings are between BALB/c ob/+ Isg15−/− and BALB/c ob/+ Isg15+/− mice. 5-8 mice of each sex and genotype are studied to assure meaningful statistical comparisons. Whole animal metabolism (food intake, energy expenditure, body composition, circulating metabolite and hormones), cellular metabolism (lipolysis rates of isolated WAT fragments) and protein expression within adipocytes (ATGL and CGI-58 steady state concentrations along with degradation rates of ATGL) are all studied.

Based on the consequences of the amino acid variation of UBE2L6:1) if the BALB/c variant of UBE2L6 abolishes or greatly reduces the its ability to perform ubiquitination, the addition of the Isg15 null allele to the BALB/c ob/ob mice will have no impact on their obesity phenotype, and 2) if the BALB/c allele of UBE2L6 greatly increases its conjugation by ISG15 and prevents UBE2L6 from participating in the ubiquitination process, then the addition of the Isg15 null allele will permit UBE2L6 to perform its role in ubiquitination of ATGL, thereby reducing ATGL concentrations, reducing lipolysis and promoting triglyceride accumulation with adipocytes.

### REFERENCES

1. Niswender K D, Schwartz M W 2003 Insulin and leptin revisited: adiposity signals with overlapping physiological and intracellular signaling capabilities. Front Neuroendocrinol 24:1-10
2. Qiu J, Ogus S, Mounzih K, Ewart-Toland A, Chehab F F 2001 Leptin-deficient mice backcrossed to the BALB/cJ genetic background have reduced adiposity, enhanced fertility, normal body temperature, and severe diabetes. Endocrinology 142:3421-3425
3. Marcelin G, Chua S, Jr. Contributions of adipocyte lipid metabolism to body fat content and implications for the treatment of obesity. Curr Opin Pharmacol 10:588-593
4. Krug R M, Zhao C, Beaudenon S 2005 Properties of the ISG15 E1 enzyme UbE1L. Methods Enzymol 398:32-40
5. Zhao C, Beaudenon S L, Kelley M L, Waddell M B, Yuan W, Schulman B A, Huibregtse J M, Krug R M 2004 The UbcH8 ubiquitin E2 enzyme is also the E2 enzyme for ISG15, an EFN-alpha/beta-induced ubiquitin-like protein. Proc Natl Acad Sci USA 101:7578-7582
6. Harty R N, Pitha P M, Okumura A 2009 Antiviral activity of innate immune protein ISG15. J Innate Immun 1:397-404
7. van Wijk S J, Timmers H T The family of ubiquitin-conjugating enzymes (E2s): deciding between life and death of proteins. FASEB J 24:981-993
8. Durfee L A, Lyon N, Seo K, Huibregtse J M The ISG15 conjugation system broadly targets newly synthesized proteins: implications for the antiviral function of ISG15. Mol-Cell 38:722-732
9. Chopra M, Galbraith S, Darnton-Hill I 2002 A global response to a global problem: the epidemic of overnutrition. Bull World Health Organ 80:952-958

10. Flynn M A, McNeil D A, Maloff B, Mutasingwa D, Wu M, Ford C, Tough S C 2006 Reducing obesity and related chronic disease risk in children and youth: a synthesis of evidence with ‘best practice’ recommendations. Obes Rev 7 Suppl 1:7-66
11. Bonow R O, Smaha L A, Smith S C, Jr., Mensah G A, Lenfant C 2002 World Heart Day 2002: the international burden of cardiovascular disease: responding to the emerging global epidemic. Circulation 106:1602-1605
12. Buettner C, Muse E D, Cheng A, Chen L, Scherer T, Pocai A, Su K, Cheng B, Li X, Harvey-White J, Schwartz G J, Kunos G, Rossetti L 2008 Leptin controls adipose tissue lipogenesis via central, STAT3-independent mechanisms. Nat Med 14:667-675
13. Holloszy J O, Kohn W M, Hansen P A 1998 The regulation of carbohydrate and fat metabolism during and after exercise. Front Biosci 3:D1011-1027
14. Villena J A, Roy S, Sarkadi-Nagy E, Kim K H, Sul H S 2004 Desnutrin, an adipocyte gene encoding a novel patatin domain-containing protein, is induced by fasting and glucocorticoids: ectopic expression of desnutrin increases triglyceride hydrolysis. J Biol Chem 279:47066-47075
15. Esposito F, Pierantoni G M, Battista S, Melillo R M, Scala S, Chieffi P, Fedele M, Fusco A 2009 Interaction between HMGA I and retinoblastoma protein is required for adipocyte differentiation. J Biol Chem 284:25993-26004
16. Wang M Y, Grayburn P, Chen S, Ravazzola M, Orci L, Unger RH 2008 Adipogenic capacity and the susceptibility to type 2 diabetes and metabolic syndrome. Proc Natl Acad Sci USA 105:6139-6144
17. Tansey J T, Sztalryd C, Gruia-Gray J, Roush D L, Zee J V, Gavrilova O, Reitman M L, Deng C X, Li C, Kimmel A R, Londos C 2001 Perilipin ablation results in a lean mouse with aberrant adipocyte lipolysis, enhanced leptin production, and resistance to diet-induced obesity. Proc Natl Acad Sci USA 98:6494-6499
18. Kajimura S, Seale P, Tomaru T, Erdjument-Bromage H, Cooper M P, Ruas J L, Chin S, Tempst P, Lazar M A, Spiegelman B M 2008 Regulation of the brown and white fat gene programs through a PRDM16/CtBP transcriptional complex. Genes Dev 22:1397-1409
19. Singh R, Xiang Y, Wang Y, Baikati K, Cuervo A M, Luu Y K, Tang Y, Pcssin J E, Schwartz G J, Czaja M J 2009 Autophagy regulates adipose mass and differentiation in mice. J Clin Invest 119:3329-3339
20. Ahmadian M, Duncan R E, Varady K A, Frasson D, Hellerstein M K, Birkenfeld A L, Samuel V T, Shulman G I, Wang Y, Kang C, Sul H S 2009 Adipose overexpression of desnutrin promotes fatty acid use and attenuates diet-induced obesity. Diabetes 58:855-866
21. Haemmerle G, Lass A, Zimmermann R, Gorkiewicz G, Meyer C, Rozman J, Heldmaicr G, Maier R, Theussl C, Eder S, Kratky D, Wagner E F, Klingenspor M, Hoefler G, Zechner R 2006 Defective lipolysis and altered energy metabolism in mice lacking adipose triglyceride lipase. Science 312:734-737
22. Lass A, Zimmermann R, Haemmerle G, Riederer M, Schoiswohl G, Schweiger M, Kienesberger P, Strauss J G, Gorkiewicz G, Zechner R 2006 Adipose triglyceride lipase-mediated lipolysis of cellular fat stores is activated by CGI-58 and defective in Chanarin-Dorfman Syndrome. Cell Metab 3:309-319
23. Hochstrasscr M 2009 Origin and function of ubiquitin-like proteins. Nature 458:422-429
24. Zechner R, Kienesberger P C, Haemmerle G, Zimmermann R, Lass A 2009 Adipose triglyceride lipase and the lipolytic catabolism of cellular fat stores. J Lipid Res 50:3-21
25. Reid B N, Ables G P, Otlivanchik O A, Schoiswohl G, Zechner R, Blaner W S, Goldberg I J, Schwabe R F, Chua S C, Jr., Huang L S 2008 Hepatic overexpression of hormone-sensitive lipase and adipose triglyceride lipase promotes fatty acid oxidation, stimulates direct release of free fatty acids, and ameliorates steatosis. J Biol Chem 283:13087-13099
26. Luo N, Liu S M, Liu H, Li Q, Xu Q, Sun X, Davis B, Li J, Chua S, Jr. 2006 Allelic variation on chromosome 5 controls beta-cell mass expansion during hyperglycemia in leptin receptor-deficient diabetes mice. Endocrinology 147:2287-2295
27. Belteki G, Haigh J, Kabacs N, Haigh K, Sison K, Costantini F, Whitsett J, Quaggin S E, Nagy A 2005 Conditional and inducible transgene expression in mice through the combinatorial use of Cre-mediated recombination and tetracycline induction. Nucleic Acids Res 33:c51
28. Srinivas S, Watanabe T, Lin C S, William C M, Tanabe Y, Jessell T M, Costantini F 2001 Cre reporter strains produced by targeted insertion of EYFP and ECFP into the ROSA26 locus. BMC Dev Biol 1:4
29. Yu H M, Liu B, Chiu S Y, Costantini F, Hsu W 2005 Development of a unique system for spatiotemporal and lineage-specific gene expression in mice. Proc Natl Acad Sci USA 102:8615-8620
30. Schuster-Gossler K, Lee A W, Lerner C P, Parker H J, Dyer V W, Scott V E, Gossler A, Conover J C 2001 Use of coisogenic host blastocysts for efficient establishment of germline chimeras with C57BL/6J ES cell lines. Biotechniques 31:1022-1024, 1026
31. Wang Z V, Deng Y, Wang Q A, Sun K, Scherer P E Identification and characterization of a promoter cassette conferring adipocyte-specific gene expression. Endocrinology 151:2933-2939
32. Sassmann A, Offermanns S, Wettschureck N Tamoxifen-inducible Cre-mediated recombination in adipocytes. Genesis: 1-8
33. de Luca C, Kowalski T J, Zhang Y, Elmquist J K, Lee C, Kilimann M W, Ludwig T, Liu S M, Chua S C, Jr. 2005 Complete rescue of obesity, diabetes, and infertility in db/db mice by neuron-specific LEPR-B transgenes. J Clin Invest 115:3484-3493
34. van de Wall E, Leshan R, Xu A W, Balthasar N, Coppari R, Liu S M, Jo Y H, MacKenzie R G, Allison D B, Dun N J, Elmquist J, Lowell B B, Barsh G S, de Luca C, Myers M G, Jr., Schwartz G J, Chua S C, Jr. 2008 Collective and individual functions of leptin receptor modulated neurons controlling metabolism and ingestion. Endocrinology 149:1773-1785
35. Butler A A, Kozak L P A recurring problem with the analysis of energy expenditure in genetic models expressing lean and obese phenotypes. Diabetes 59:323-329
36. Leiter E H, Coleman D L, Eisenstein A B, Strack 11980 A new mutation (db3J) at the diabetes locus in strain 129/J mice. I. Physiological and histological characterization. Diabetologia 19:58-65
37. Osiak A, Utermohlen O, Niendorf S, Horak I, Knobeloch K P 2005 ISG15, an interferon-stimulated ubiquitin-like protein, is not essential for STAT1 signaling and responses against vesicular stomatitis and lymphocytic choriomeningitis virus. Mol Cell Biol 25:6338-6345
38. Skaug B, Chen Z J Emerging role of ISG15 in antiviral immunity. Cell 143:187-190
39. Ceccarelli D F et al., An allosteric inhibitor of the human Cdc34 ubiquitin-conjugating enzyme. Cell. 2011 Jun. 24; 145(7):1075-87.

1. A method for treating obesity, treating an obesity comorbidity, or treating a dyslipidemia in a subject comprising administering to the subject an amount of an inhibitor of an E2 ubiquitin ligase activity effective to treat the obesity, obesity comorbidity, or dyslipidemia in the subject.

2. A method for treating obesity or a dyslipidemia in a subject comprising administering to the subject an amount of an activator of adipocyte triglyceride lipase, or an enhancer of adipocyte triglyceride lipase activity, effective to treat the obesity or dyslipidemia in the subject.

3. (canceled)

4. The method of claim 1, wherein the E2 ubiquitin ligase is UBE2L6.

5. The method of claim 1, wherein the inhibitor of E2 ubiquitin ligase is an antibody or a fragment of an antibody.

6. The method of claim 5, wherein the antibody is a monoclonal antibody or the fragment of an antibody is a fragment of a monoclonal antibody.

7. The method of claim 1, wherein the inhibitor of E2 ubiquitin ligase is an shRNA or siRNA directed to UBE2L6 or a small molecule inhibitor of UBE2L6 of 200 daltons or less.

8. The method of claim 2, wherein the enhancer of adipocyte triglyceride lipase activity is a UBE2L6 inhibitor.

9. The method of claim 8, wherein the UBE2L6 inhibitor is an antibody or fragment of an antibody.

10. The method of claim 9, wherein the antibody is a monoclonal antibody or the fragment of an antibody is a fragment of a monoclonal antibody.

11. The method of any of claim 1, wherein the method is for treating obesity.

12. A method for identifying an agent as a treatment for obesity or as a candidate agent for treating obesity comprising:
a) contacting a protein with UBE2L6 under conditions permitting ubiquitination of the protein by the UBE2L6;
b) quantitating the ubiquitination of the protein by the UBE2L6;
c) contacting the UBE2L6 with the agent; and
d) quantitating the ubiquitination of the protein by the UBE2L6 in the presence of the agent,
wherein a decreased ubiquitination of the protein by the UBE2L6 in the presence of the agent indicates that the agent is a treatment for obesity or is a candidate agent for treating obesity and wherein no change in or an increased ubiquitination of the protein by the UBE2L6 on the protein in the presence of the agent indicates that the agent is a treatment for obesity or is a candidate agent for treating obesity.

13. The method of claim 12, wherein the protein is adipose triglyceride lipase.

14. (canceled)

15. The method of claim 12, wherein the method is performed in vitro.

16. The method of claim 12, wherein the agent is an organic molecule of 2000 daltons or less, an antibody, an oligonucleotide, or an shRNA or an siRNA.

17-27. (canceled)


SEQUENCE LISTING (1 sequence)

SEQ ID NO: 1 - protein, 153 aa, HOMO SAPIENS
MMASMRVVKELEDLQKKPPPYLRNLSSDDANVLVWHALLLPDQPPYHLKAFNLRISFPPEYPFKPPMIKFTTKIYHPNVDENGQICLPIISSENWKPCTKTCQVLEALNVLVNRPNIREPLRMDLADLLTQNPELFRKNAEEFTLRFGVDRPS